(19) United States
(12) Patent Application Publication
(10) Pub. No.: US 2009/0018592 A1
(43) Pub. Date: Jan. 15, 2009

(54) BONE SCREW FOR ORTHOPEDIC APPARATUS

(76) Inventor: Richard B. Pitbladdo, Naples, FL (US)

Correspondence Address: BROWN & MICHAELS, PC;400 M & T BANK BUILDING, 118 NORTH TIOGA ST, ITHACA, NY, 14850, US

(21) Appl. No.: 12/170,950

(22) Filed: Jul. 10, 2008

Related U.S. Application Data
(60) Provisional application No. 60/949,596, filed on Jul. 13, 2007.

(51) Int. Cl. A61B 17/04; A61F 2/28

(52) U.S. Cl. 606/309; 606/301; 623/16.11

(57) ABSTRACT

A bone screw deforms on the same order as the bone, thus providing substantially uniform loading along an entire length of the thread of the bone screw. The bone screw of the present invention evenly distributes stress by matching the effective cross-sectional area of the bone screw times its modulus of elasticity with the effective cross-sectional area of the parent material (i.e. bone) times its modulus of elasticity so that they are preferably substantially equal to each other.

### REFERENCE TO RELATED APPLICATIONS

This application claims one or more inventions which were disclosed in Provisional Application No. 60/949,596, filed Jul. 13, 2007, entitled “BONE SCREW FOR ORTHOPEDIC APPARATUS”. The benefit under 35 USC §119(e) of the United States provisional application is hereby claimed, and the aforementioned application is hereby incorporated herein by reference.

### BACKGROUND OF THE INVENTION

1. Field of the Invention

The invention pertains generally to the design of screws to secure orthopedic reinforcements to bones.

2. Description of Related Art

The attachments of orthopedic reinforcements to bone are limited by how well the screws inserted into the bone secure the reinforcements. It has been determined that the attachment strength of a screw with a few (2-3) threads is substantially as strong as one screw with many (4 or more) threads. Using a longer standard screw versus a short standard screw makes little difference in the attachment strength. This is a major drawback of present practice in the applications where attachment strength is important.

### SUMMARY OF THE INVENTION

The present invention provides bone screw designs whereby the screw deforms on the same order as the bone, thus providing substantially uniform loading along a substantial portion of the length of a thread of the bone screw. The lengthwise tension loading is much more uniform than the tension loading in the prior art. In fact, some of the embodiments of the present invention provide substantially uniform loading along the entire length of the thread.

A bone screw of the present invention evenly distributes stress by matching the effective cross-sectional area of the bone screw times its modulus of elasticity with the effective cross-sectional area of the parent material (i.e. bone) times its modulus of elasticity so that they are preferably substantially equal to each other.

### BRIEF DESCRIPTION OF THE DRAWINGS

FIG. 1 shows a prior art bone screw.

FIG. 2 shows a bone screw with a hollow center in an embodiment of the present invention.

FIG. 3 shows a helical bone screw in an embodiment of the present invention.

FIG. 4 shows a bone screw with a tapered hollow center in an embodiment of the present invention.

FIG. 5 shows a tapered helical bone screw in an embodiment of the present invention.

FIG. 6 shows a tapered insert for the tapered helical bone screw of FIG. 5 in an embodiment of the present invention.

FIG. 7 shows a helical bone screw with a center reinforcement insert in an embodiment of the present invention.

FIG. 8A shows a tapered insert for the tapered helical bone screw of FIG. 7 in an embodiment of the present invention.

FIG. 8B shows a tapered insert for the tapered helical bone screw of FIG. 7 in another embodiment of the present invention.

FIG. 8C shows a tapered insert for the tapered helical bone screw of FIG. 7 in another embodiment of the present invention.

FIG. 9 shows a bone screw with a hollow center over a portion of its length in an embodiment of the present invention.

### DETAILED DESCRIPTION OF THE INVENTION

A standard screw made of stainless steel or titanium is much more rigid than the bone into which it is inserted. When a lengthwise/longitudinal tension load is applied to the screw, the bone deforms much more readily than the screw. The loading of the bone screw is not at all uniform along a length of the thread. Thus, only a few sections of the thread of the screw support the entire load. If the load on the bone is too high at these sections, the bone yields to failure at these sections and transfers the stress to other sections of the thread. Thus, there is a cascading phenomenon until the bone at all sections of the thread fails and the attachment is broken.

The present invention includes bone screws with different configurations in various embodiments. There are two different groups of embodiments. In one group of embodiments, the shape of the bone screw is varied to decrease the total cross-sectional area of the bone screw. In another group of embodiments, the material from which the bone screw is made has a much lower modulus of elasticity than titanium or stainless steel, the materials currently used for bone screws. The shape embodiments use screws preferably made from present materials (such as stainless steel or titanium), whereas the material embodiments require a material with mechanical properties matched to the bone material by the relationship discussed herein. Both groups of embodiments evenly distribute stress by matching the effective cross-sectional area of the screw times its modulus of elasticity with the effective cross-sectional area of the parent material (i.e. bone) times its modulus of elasticity so that they are preferably substantially equal to each other.

Stress analysis of a screw inserted into a parent material shows that the screw may be made from a material which is substantially stiffer than the parent material. To create a relatively even distribution of stress from the screw threads to the parent material, the present invention substantially equates the effective cross-sectional area of the screw (Aₛ) times its modulus of elasticity (Eₛ) to the effective cross-sectional area of the parent material (Aₚ) times its modulus of elasticity (Eₚ).

(Aₛ)×(Eₛ)≈(Aₚ)×(Eₚ). (1)

This relationship equates equal and opposite forces on the screw threads along the length of the attachment. In the embodiments where all or a portion of the screw resembles a spring, the force on the screw threads can be expressed as the spring rate (Kₛ) of the thread times the length (Iₛ) of the thread.

(Kₛ)×(Iₛ)≈(Aₚ)×(Eₚ). (2)

Note that:

(Aₛ)=(Kₛ)×(Iₛ)/(Eₛ). (3)

In a preferred embodiment of the present invention, the parent material is bone.

As defined herein, the effective cross-sectional area of the bone is the cross-sectional area which is substantially deformed or displaced under load by the inserted screw. The effective cross-sectional area of the portion of the screw with a shank is either the actual cross-sectional area of the screw material when you take a cross-section perpendicular to the length of the screw at the shank or a function of the spring rate of the screw as shown in equation (3) above. The effective cross-sectional area of the portion of the screw including only one or more threads (and no shank) is a function of the spring rate of the thread as shown in equation (3) above.

In preferred embodiments of the present invention, for at least one point or section along the length of the bone screw, the effective cross-sectional area of the bone screw material times the modulus of elasticity of the screw material is substantially equal to the effective cross-sectional area of the bone times the modulus of elasticity of the bone.

In other preferred embodiments of the present invention, for at least one point or section along the length of the bone screw, the spring rate of that section of the bone screw times the length of that section of the bone screw is substantially equal to the effective cross-sectional area of the bone times the modulus of elasticity of the bone.

As an example, if a titanium screw with a modulus of elasticity (Eₛ) of 16.5×10⁶ psi and a cross sectional area (Aₛ) of 0.01 square inches was secured in a bone with an effective area (A_{b}) of 0.25 square inches and a modulus of elasticity (E_{b}) of 0.66×10⁶ psi, the load on the bone material would be substantially evenly distributed (16.5×10⁶×0.01=0.66×10⁶×0.25) along the entire length of the screw thread.

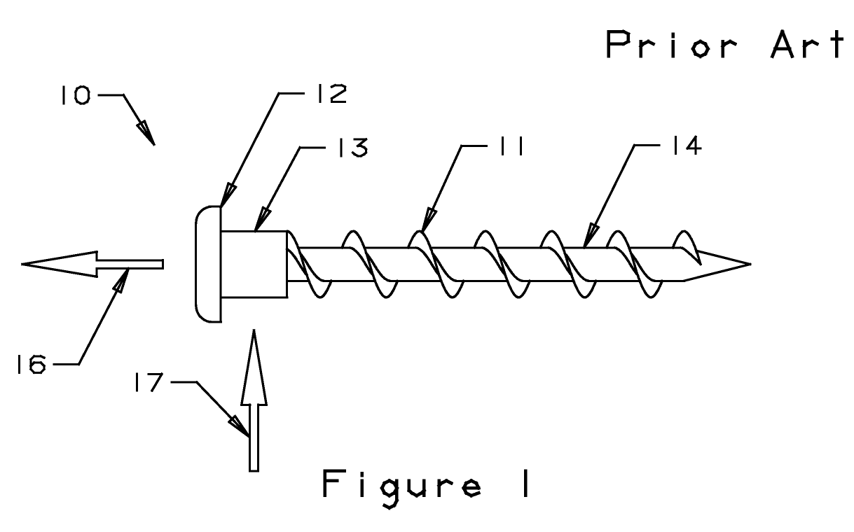

FIG. 1 shows a prior art bone screw (10). It is essentially the same as a standard button head screw. It consists of a thread (11) which is part of the shank (14) of the screw (10). The prior art bone screw (10) has a shoulder (13) where it passes through the orthopedic device to be attached to the bone and a head (12) to secure the orthopedic device to the bone. A lengthwise/longitudinal tension load (16) is applied to the screw when the screw is attached to the bone. The stresses on the attachment of an orthopedic device to a bone also include a shear force (17) perpendicular to the lengthwise/longitudinal axis of the screw. A bone screw of the present invention is preferably designed to target the longitudinal tension load (16); however, bone screws of the present invention may also be designed to have the shear and bending strength to accommodate the shear force (17).

Bone typically has a lower modulus of elasticity than the 0.66×10⁶ noted in the example. Therefore, a bone screw of titanium or stainless steel must have a lower effective cross-sectional area than the cross-sectional area of the prior art screws in order to more evenly distribute the load across the threads of the screw.

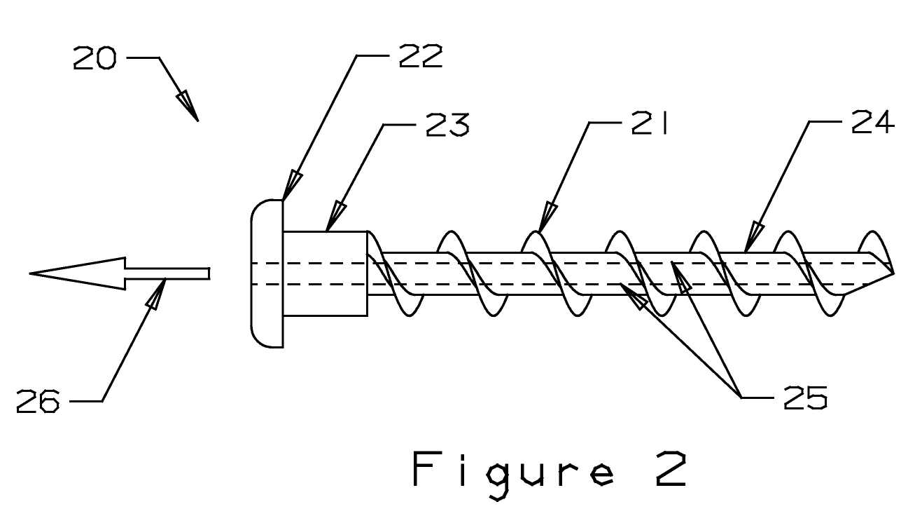

FIG. 2 shows a bone screw (20) in an embodiment of the present invention. The screw (20) has a thread (21), a shank (24), a shoulder (23), and a head (22). The bone screw (20) also has a hole (25) in the centerline of the bone screw, thus reducing its cross-sectional area. The diameter of the hole (25) is chosen to reduce the cross-sectional area of the shank (24) and the thread (21) combined, to match the relationship between the modulus of elasticity and cross-sectional area previously described. In a preferred embodiment, the bone screw (20) may be made of a material that has a high modulus of elasticity, such as titanium or stainless steel. A lengthwise (longitudinal) tension load (26) is applied to the screw when the screw is attached to the bone. In a preferred embodiment, the center cavity is filled with an insert or a paste of artificial bone.

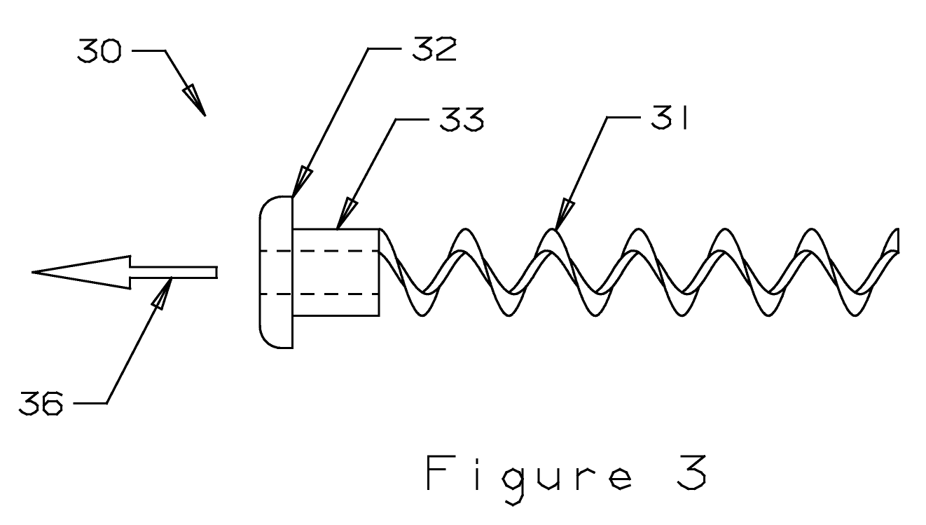

FIG. 3 shows a bone screw (30) in another embodiment of the present invention, which is the limit in reducing the effective cross-sectional area. The screw has a thread (31), a shoulder (33), and a head (32); however, it has no shank. It is in actuality a helical spring. Because the cross-sectional area has been significantly reduced, in a preferred embodiment, the bone screw (30) may be made of a material with a high modulus of elasticity, such as titanium or stainless steel. A lengthwise (longitudinal) tension load (36) is applied to the screw when the screw is attached to the bone.

Because of its flexibility, the helical bone screw (30) requires tapping of the bone prior to installation. A tap of suitable shape would be required. After insertion of the threaded helix, the center cavity is preferably filled with a plug or a paste of artificial bone.

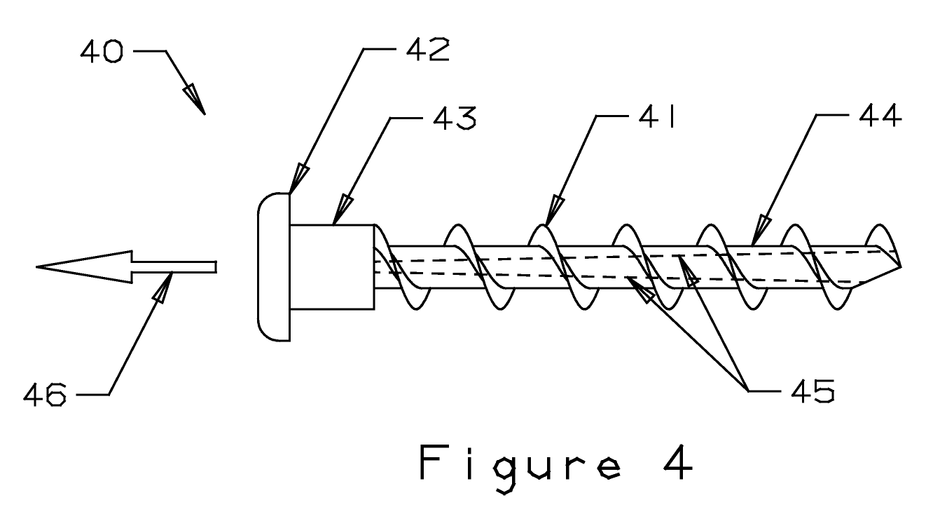

The bone screw configurations of FIGS. 2 and 3 would be ideal if the primary attachment parameter is lengthwise (longitudinal) tension and if it was possible to determine the exact modulus of elasticity of an individual's bone with accuracy. This is not presently possible considering the various effects of age and health on an individual's bone properties. To minimize the need for an exact determination of the bone properties, the embodiments of FIGS. 4, 5, 7, and 9 have a variable cross-section along the length of the bone screw.

FIG. 4 shows a screw (40) in an alternative embodiment of the present invention, which has a variable effective cross-sectional area along its length. The screw (40) has a thread (41), a shank (44), a shoulder (43), and a head (42), which are substantially the same as shown in FIG. 2. The difference between this embodiment and the embodiment shown in FIG. 2 is that the hole (45) in the centerline of the bone screw is tapered, thus varying the cross-sectional area along the length of the screw (40). A lengthwise (longitudinal) tension load (46) is applied to the screw when the screw is attached to the bone. In a preferred embodiment, the bone screw (40) may be made of a material with a high modulus of elasticity, such as titanium or stainless steel. In another preferred embodiment, the center cavity is filled with an insert or a paste of artificial bone.

This embodiment, with its range of cross-sectional area, does not give as strong an attachment as the embodiments of FIGS. 2 and 3 when these embodiments are perfectly matched to the bone elasticity; however, it allows for a large margin of mismatch to the bone elasticity and gives greatly improved stress distribution than the prior art.

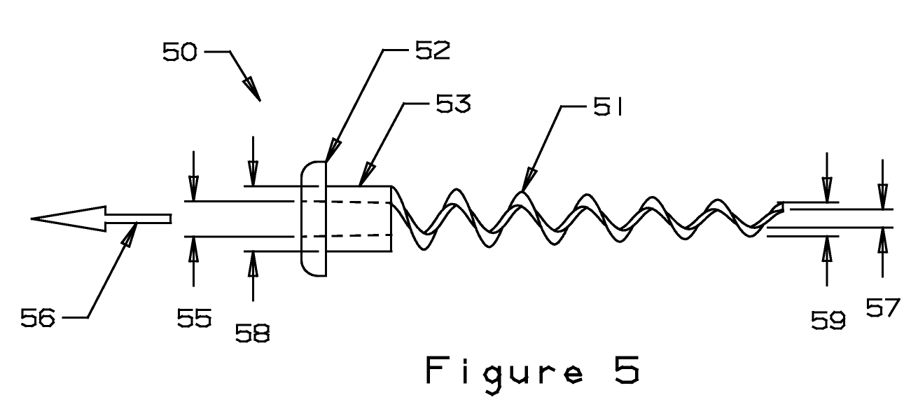

FIG. 5 shows a bone screw (50) in another embodiment of the present invention, which has a variable effective cross-sectional area along its length. The screw (50) has a shoulder (53), a head (52), and a tapered helical thread (51). The inside diameter (55) of the thread (51) at the shoulder (53) end is greater than the inside diameter (57) at the distal end. Additionally, the outside diameter (58) of the thread (51) at the shoulder (53) end is greater than the outside diameter (59) at the distal end. The difference between the outside diameter (58) and the inside diameter (55) at the shoulder (53) end is greater than the difference between the outside diameter (59) and the inside diameter (57) at the distal end. In another embodiment, the outside diameter (58) of the thread (51) at the shoulder (53) end is equal to the outside diameter (59) at the distal end. In this embodiment, the difference between the outside diameter (58) and the inside diameter (55) at the shoulder (53) end is greater than the difference between the outside diameter (59) and the inside diameter (57) at the distal end. The tapered helical shape of the bone screw has the effect of making the effective elastic modulus of the screw as a system substantially lower than a prior art bone screw. The taper may be in the thickness and/or the shape of the thread (51). In a preferred embodiment, the bone screw (50) may be made of a material with a high modulus of elasticity, such as titanium or stainless steel. A lengthwise (longitudinal) tension load (56) is applied to the screw when the screw is attached to the bone.

The tapered helical bone screw (50) shown in FIG. 5 requires tapping of the bone prior to installation. A tap of suitable shape would be required.

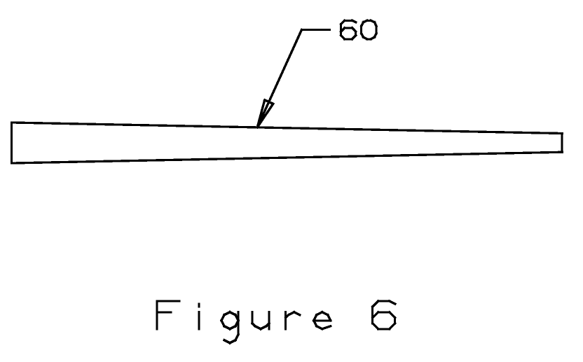

FIG. 6 shows a tapered insert (60) that is preferably inserted into the center of the tapered helical bone screw (50) shown in FIG. 5. The tapered insert (60) is preferably made of material that is compatible with bone, i.e. artificial bone. The tapered insert (60) maintains the threads in position until the bone has healed.

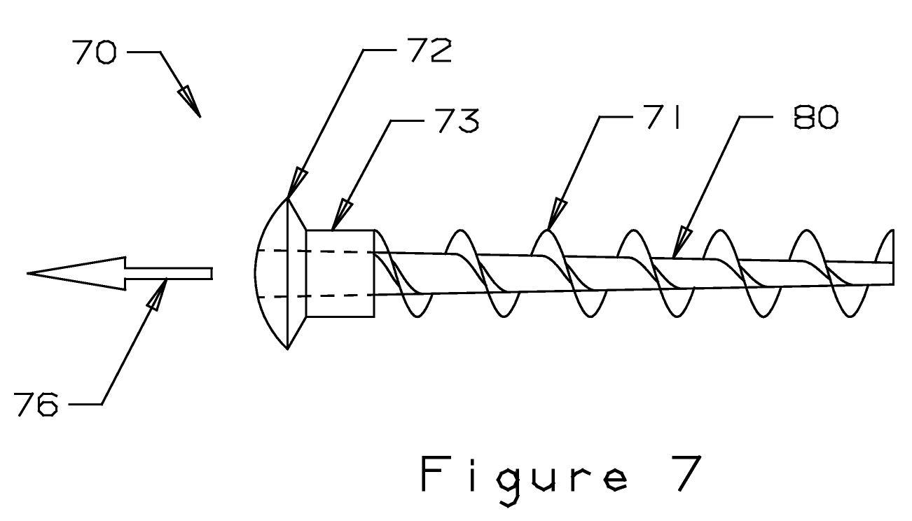

FIG. 7 shows a bone screw (70) in another embodiment of the present invention, which has a variable effective cross-sectional area along its length. The screw (70) has a shoulder (73), a head (72), and a helical shape of the thread (71). The head (72) shown in FIG. 7 is a countersunk head. A lengthwise (longitudinal) tension load (76) is applied to the screw when the screw is attached to the bone. A tapered center insert (80) is inserted into the center of the helical shaped thread (71). In a preferred embodiment, the bone screw (70) may be made of a material with a high modulus of elasticity, such as titanium or stainless steel.

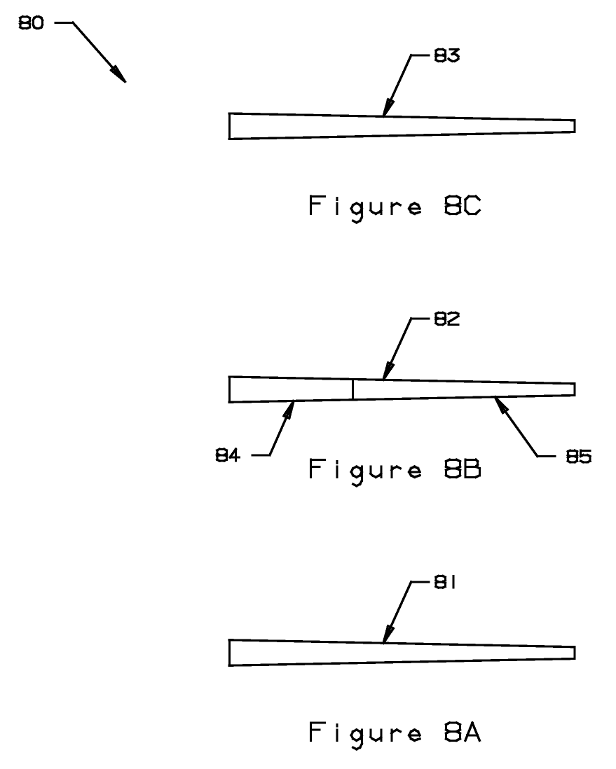

FIGS. 8A through 8C show three configurations of a tapered insert (80) that is preferably inserted into the center of the tapered helical bone screw (70) shown in FIG. 7. The tapered insert (81) shown in FIG. 8A is preferably made from a material with a high modulus of elasticity, such as titanium or stainless steel. The tapered insert (82) shown in FIG. 8B is made from two materials. The head end (84) of the insert (82) is made from a material with a high modulus of elasticity, such as titanium or stainless steel, and the distal end (85) is made from artificial bone material. The inserts (81) and (82) act in the same manner as the shank of the prior art bone screw (10) by providing an increase in the shear and bending strength of the bone screw; however, these inserts (81) and (82) do not change its longitudinal elasticity. The tapered insert (83) shown in FIG. 8C is made from artificial bone material. All of the configurations (81), (82) and (83) of the tapered insert (80) maintain the threads in position until the bone has healed. All of these configurations could alternatively be used as inserts for the bone screw shown in FIG. 5.

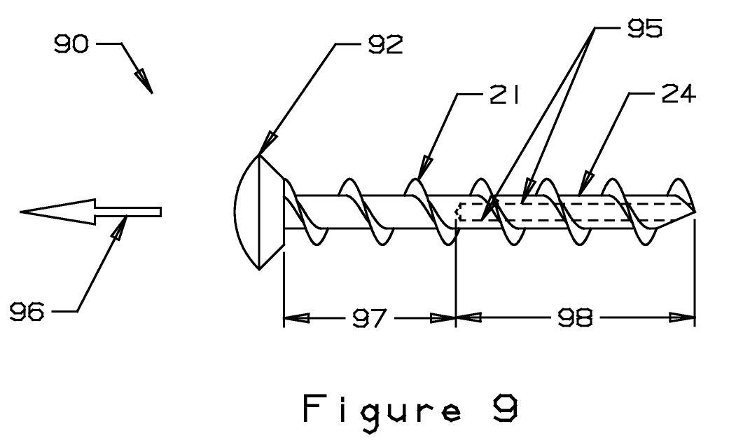

FIG. 9 shows an embodiment similar to the embodiment shown in FIG. 2, except that the center hole (95) has a depth (98) that does not extend the entire length of the screw (90) and the head configuration is different. A lengthwise (longitudinal) tension load (96) is applied to the screw when the screw is attached to the bone. In this embodiment, the bone screw (90) has substantially the same shear and bending strength as the screw (10) of the prior art because of the length of the solid portion (97) of the shank (24). This embodiment also has the advantages of the lengthwise tensile properties of the present invention because of the lower cross-sectional area of the shank (24) section (98) containing the hole (95). The head (92) configuration has no shoulder and is countersunk to match the orthopedic apparatus. In another preferred embodiment, the center cavity is filled with an insert or a paste of artificial bone.

FIGS. 1, 2, 3, and 5 show a screw with a button head and a shoulder. FIG. 7 shows a screw with a countersunk head and a shoulder. FIG. 9 shows a screw with a countersunk head only. These represent three of the many head attachment configurations that would be matched to the design of the orthopedic apparatus. Other head configurations are also within the spirit of the present invention.

While all of the bone screws illustrated herein have single threads, the same principles of this invention would apply to screws configured with two or more threads.

In another embodiment of the present invention, the bone screw is made of a material which, when formed into a screw, has a modulus of elasticity substantially lower than that of stainless steel or titanium; such that when the material is formed into a screw, the cross-sectional area and modulus of elasticity relationship described above is obtained. With this material, the load is more evenly distributed along a length of the thread of the screw, thus strengthening the screw bone attachment system. In other embodiments, a material with a lower modulus of elasticity than titanium or stainless steel may be used in combination with any of the bone screws shown in the embodiments of FIGS. 2, 4, 5, 7 and 9 to create a relatively even distribution of stress along a length of the bone screw thread to the parent material, by equating the effective cross-sectional area of the screw times its modulus of elasticity to the effective cross-sectional area of the bone times its modulus of elasticity.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A bone screw apparatus comprising a bone screw, wherein an effective cross-sectional area of the bone screw times a modulus of elasticity of the bone screw matches an effective cross-sectional area of a bone times a modulus of elasticity of the bone such that the bone deforms on a same order as the bone screw when the bone screw is attached to the bone.

2. The apparatus of claim 1, wherein the bone screw is made of a material that has a substantially lower modulus of elasticity than stainless steel or titanium.

3. The apparatus of claim 1, wherein the effective cross-sectional area of the bone screw times the modulus of elasticity of the bone screw is substantially equal to the effective cross-sectional area of the bone times the modulus of elasticity of the bone.

4. The apparatus of claim 1, further comprising an artificial bone material that fills any internal voids in the bone screw.

5. The apparatus of claim 1, wherein the bone screw has a variable cross-sectional area along at least a portion of a length of the bone screw.

6. The apparatus of claim 1, wherein the bone screw comprises:
a) a head;
b) a shoulder connected to the head; and
c) at least one thread extending from the shoulder, wherein there is substantially uniform loading along a substantial portion of a length of the thread of the bone screw when the bone screw is attached to the bone.

7. The apparatus of claim 6, wherein there is substantially uniform loading along an entire length of the thread.

8. The apparatus of claim 6, further comprising an insert placed into a center of the bone screw.

9. The apparatus of claim 8, wherein the insert is made of stainless steel or titanium.

10. The apparatus of claim 8, wherein the insert is made of a combination of stainless steel or titanium and artificial bone.

11. The apparatus of claim 8, wherein the insert is made of artificial bone.

12. The apparatus of claim 6, wherein the bone screw further comprises a shank extending from the shoulder, wherein the thread comprises a threaded portion of a shank.

13. The apparatus of claim 12, wherein the bone screw further comprises a hollow inner portion along a lengthwise direction of the bone screw.

14. The apparatus of claim 13, wherein the hollow inner portion is tapered in the lengthwise direction of the bone screw.

15. The apparatus of claim 13, wherein the hollow inner portion has a depth less than an entire length of the bone screw.

16. The apparatus of claim 13, further comprising an artificial bone material that fills the hollow inner portion.

17. The apparatus of claim 1, wherein the bone screw comprises:
a) a head; and
b) at least one thread extending from the head, wherein there is substantially uniform loading along a substantial portion of a length of the thread of the bone screw when the bone screw is attached to the bone.

18. The apparatus of claim 17, further comprising an insert placed into a center of the bone screw.

19. The apparatus of claim 18, wherein the insert is made of stainless steel or titanium.

20. The apparatus of claim 18, wherein the insert is made of a combination of stainless steel or titanium and artificial bone.

21. The apparatus of claim 18, wherein the insert is made of artificial bone.

22. The apparatus of claim 17, wherein the bone screw further comprises a shank extending from the head, wherein the thread comprises a threaded portion of a shank.

23. The apparatus of claim 22, wherein the bone screw further comprises a hollow inner portion along a lengthwise direction of the bone screw.

24. The apparatus of claim 23, wherein the hollow inner portion is tapered in the lengthwise direction of the bone screw.

25. The apparatus of claim 23, wherein the hollow inner portion has a depth less than an entire length of the bone screw.

26. The apparatus of claim 23, further comprising an artificial bone material that fills the hollow inner portion.

27. The apparatus of claim 1, wherein, for at least one point along a length of the bone screw, the effective cross-sectional area of a material of the bone screw times the modulus of elasticity of the material is substantially equal to the effective cross-sectional area of the bone times the modulus of elasticity of the bone.

28. The apparatus of claim 1, wherein, for at least one section along a length of the bone screw, a spring rate of the section of the bone screw times the length of the section of the bone screw is substantially equal to the effective cross-sectional area of the bone times the modulus of elasticity of the bone.

29. A method of evenly distributing stress on a bone screw in an orthopedic device, comprising the step of inserting a bone screw into at least a portion of a bone, wherein the bone screw deforms substantially a same amount as the bone when force is applied to the bone and the bone screw.

30. The method of claim 29, further comprising the step of providing substantially uniform loading along a substantial portion of a length of a thread of the bone screw when the bone screw is inserted into the bone.

31. The method of claim 29, further comprising the step of providing substantially uniform loading along an entire length of a thread of the bone screw when the bone screw is inserted into the bone.

32. The method of claim 29, wherein the bone screw has an effective cross-sectional area times a modulus of elasticity that matches an effective cross-sectional area of the bone times a modulus of elasticity of the bone such that the bone deforms on a same order as the bone screw when the bone screw is attached to the bone.

33. A method of evenly distributing stress in a bone screw inserted into a bone comprising the step of designing a bone screw having an effective cross-sectional area times a modulus of elasticity of the bone screw that matches an effective cross-sectional area of the bone times a modulus of elasticity of the bone such that the bone deforms on a same order as the bone screw when the screw is attached to the bone.

34. The method of claim 33, wherein the effective cross-sectional area times the modulus of elasticity of the bone screw is substantially equal to the effective cross-sectional area of the bone times the modulus of elasticity of the bone.
